(19) United States
(12) Patent Application Publication
(10) Pub. No.: US 2012/0265072 A1
(43) Pub. Date: Oct. 18, 2012

(54) METHODS AND SYSTEMS FOR THE ULTRASOUND GUIDED SUB-FACIAL SKELETAL MUSCLE INJECTION OF AUTOLOGOUS FAT TO PRODUCE MUSCLE VOLUME EXPANSION

(76) Inventor: David L. Matlock, Los Angeles, CA (US)

(21) Appl. No.: 13/448,314

(22) Filed: Apr. 16, 2012

Related U.S. Application Data
(60) Provisional application No. 61/476,177, filed on Apr. 15, 2011.

(51) Int. Cl. A61M 37/00; A61K 35/12; A61M 1/00

(52) U.S. Cl. 600/439; 604/542; 424/574

(57) ABSTRACT

The method includes novel applications for liposuction, lipectomy, liposculpting, and ultrasonic fat harvesting and emulsification devices, especially those devices that provide in situ visualization. The application includes immediately transferring harvested autologous fat from the liposuction, lipectomy, liposculpting, or ultrasonic device to an injector portion on the fat harvesting and emulsifying device or to a separate injector unit that injects it into muscle to provide a higher value, more desirable, use for and positioning of the fat in the form of muscle volume expansion.

### CROSS-REFERENCES TO RELATED APPLICATIONS

This application claims priority from and is a nonprovisional of U.S. provisional Application No. 61/476,177 filed Apr. 15, 2011 which is herein incorporated by reference in its entirety.

### BACKGROUND

This invention relates generally to new applications for an ultrasound guided fat emulsification unit with visualization, and more particularly concerns the application of using such a unit to provide muscle volume expansion from injection of autologous fat.

Many individuals have excess fat in unwanted places on the body while lacking muscle tone and definition in other desired places. It is not uncommon for such individuals with the means to seek out the services of a skilled plastic or cosmetic surgeon to assist in transforming their physique in ways that diet and exercise cannot. Often times the excised fat from liposuction and lipectomy procedures goes to waste and is simply disposed of hygienically in a biohazard receptacle. It would be desirable to provide an immediate beneficial use for some of the excised fat on the same patient seeking muscle enhancement in locations on the body other than those from which the fat was removed. The present invention solves this problem and provides methods for immediate use of such excised fat to provide muscle volume expansion.

Current state of the art ultrasound guided fat emulsification units now provide visualization during liposuction and lipectomy procedures. This capability is currently unexploited and may be especially advantageous for subsequent use of the removed emulsified fat to be selectively injected sub-facially into certain skeletal muscles for muscle volume expansion. The present invention solves this problem and provides a high value application for an ultrasound guided fat emulsification unit with visualization.

Many individuals lacking adequate muscle tone feel self-conscious about their appearance. This may particularly be the case for patients battling diseases and conditions that are characterized by muscle wasting, including HIV, AIDS, cancer, etc. Such patients are not always physically able to exercise as needed to rebuild and restore muscle volume. It would be desirable to provide a fat injection technique to the muscles of such individuals to restore a healthy appearance and increase self-confidence. In such cases the individuals afflicted with such conditions or diseases may not always have sufficient adipose tissue to harvest for injection into the muscles. Accordingly, in these circumstances the adipose tissue may be generated ex vivo, in vitro from stem cells or else may be nonautologous.

The present invention solves these problems and addresses these and other needs.

### SUMMARY OF THE INVENTION

Provided herein are methods and systems for the autologous subfascial skeletal muscle injection of processed adipose tissue and or adipose derived stem cells from lipoaspirate or excisional lipectomy within any skeletal muscle tissue beneath the myofascia and directly within muscle tissue under direct ultrasound guidance or through a blind technique. The injection may be performed with any type of injection cannula, needle, or other instrument. Adipose tissue may be processed mechanically or manually and may be chemically treated or untreated.

Any of the skeletal muscles of the human muscular system can be treated according to the methods of the present invention, including but not limited to the biceps brachii, triceps brachii, deltoids, brachialis, deltoid, pectorals major, rectus abdominus, abdominal external oblique, quadriceps femoris, pronator teres, brachioradialis, trapezius, latissimus dorsi, biceps femoris, soleus, levator scapulae, gluteus maximus, and the like.

Adipose tissue is commonly acquired by a liposuction or lipectomy procedure in an open or closed system. Adipose tissue so acquired can then be washed and purified to increase the adipose content and/or adipose derived stem cells for use as an autologous injectable filler. The liposuction techniques that may be used to acquire the adipose tissue for subsequent use as autologous injectable filler include, but are not limited to: suction assisted lipoplasty, dry technique, wet technique, super wet technique, tumescent technique, ultrasound assisted liposuction, external ultrasound assistance, and the like.

The cannulas used in liposuction or lipectomy procedures for acquiring adipose tissue may be of static nonmechanical or power-assisted liposuction systems. The liposuction procedures included herein, prior to the sub-fascial skeletal muscle injection of the harvested fat, may be performed under local anesthesia, local with intravenous sedation, epidural, spinal, twilight or general anesthesia.

Autologous fat is the patient's own fat and is living tissue and remains alive if it becomes revascularized in a transplanted area. The methods and systems described herein may also include the usage of Adipose Derived Regenerative Cells (ADRC) as injectable filler for muscle volume expansion.

### DETAILED DESCRIPTION OF THE PREFERRED EMBODIMENTS

The methods and systems covered herein can be used to aesthetically enhance some or all of the skeletal muscle groups to produce a more athletic muscular appearance of a male or female or to augment muscle groups of individuals who are already muscular such as body builders (amateur or professional).

The methods and systems can also be used for individuals with muscle wasting diseases such as HIV.

The methods and systems also include the use of fat that has been banked and stored for future use.

The methods and systems also include regenerative engineering of fat, for example Adipose Derived Regenerative Cells (ADRCs) used to grow fat cells or muscles to be re-implanted into the donor or non donor. The methods and systems include stem cells purified from any bodily source such as bone marrow or embryonic stem cells and all tissue from excisional lipectomy are included. Additionally, non-autologous fat fillers like hyaluronics or oil derivatives are included as within the scope and for use with the methods of the present invention.

Delivery with blunt or sharp cannulaes, hypodermic needles with syringes or specialized delivery mechanisms such as fat injection guns are included.

Injection with ultrasound guidance or blindly are included.

Muscle fascia is a connective tissue sheath firmly adhered to the underlying muscle and is thought to minimize reduction of muscular force by minimizing friction during muscle movement. Muscle fascia may divide muscle groups into separate bundles by intramuscular septa. Myofascia (muscle fascia) has limited pliability and may act as a limiting agent in muscle hypertrophy.

The injection of processed autologous material with either subfascial or intra muscular placement or both would have potential muscle effects, including but not limited to:

(1) The revascularization and retention of processed adipose cells to provide an apparent hypertrophy of the muscle tissue;

(2) The non-embryonic stem cells normally found in processed adipose tissue forming new myocytes (muscle cells) resulting in muscle hyperplasia;

(3) A mass effect of the subfascial filler resulting in enhanced myofasial pliability allowing for increased muscle hypertrophy if subject is in a proper anabolic state; and

(4) Any combination of the above effects.

I Claim:

1. A method of expanding a muscle's volume, comprising:
injecting autologous fat into the muscle.

2. A method of expanding a muscle's volume as in claim 1, further comprising:
harvesting autologous fat from a patient's body with a lipectomy device.

3. A method of expanding a muscle's volume as in claim 1, further comprising:
harvesting autologous fat from a patient's body with a liposuction device.

4. A method of expanding a muscle's volume as in claim 1, further comprising:
harvesting autologous fat from a patient's body with an ultrasonic device that also emulsifies fat; and
emulsifying autologous fat from the patient's body with the ultrasonic device as or after it is harvested.

5. A method of expanding a muscle's volume as in claim 4, wherein the ultrasonic device provides visualization during fat harvesting.

6. A method of expanding a muscle's volume as in claim 5, wherein injecting autologous fat into a muscle occurs after the fat is harvested.

7. A method of expanding a muscle's volume as in claim 6, wherein injecting is performed by an injector portion on the ultrasonic device.

8. A method of expanding a muscle's volume as in claim 6, wherein injecting is performed by an injector unit attachable to the ultrasonic device.

9. A method of expanding a muscle's volume as in claim 5, further comprising:
transferring emulsified autologous fat harvested and emulsified by the ultrasonic device from the ultrasonic device to a separate injector device before it is injected into the muscle.

10. A method of expanding a muscle's volume as in claim 7, further comprising:
transferring emulsified autologous fat harvested and emulsified by the ultrasonic device to the injector portion on the ultrasonic device, before it is injected into the muscle, with a transfer unit connected to the injector portion of the ultrasonic device.

11. A method of expanding a muscle's volume as in claim 8, further comprising:
transferring emulsified autologous fat harvested and emulsified by the ultrasonic device to the injector unit attachable to the ultrasonic device, before it is injected into the muscle, with a transfer unit attachable to both the injector unit and the ultrasonic device.

12. A method of expanding a muscle's volume as in claim 1, wherein a source of the autologous fat is Adipose Derived Regenerative Cells (ADRC).

13. A method of expanding a muscle's volume as in claim 1, wherein the muscle is a bicep.

14. A method of expanding a muscle's volume as in claim 1, wherein the muscle is a tricep.

15. A method of expanding a muscle's volume comprising:
injecting fat sub-fascially into a skeletal muscle using an ultrasound guided device that provides direct visualization.

16. A method of expanding a muscle's volume as in claim 15, wherein the skeletal muscle is a bicep.

17. A method of expanding a muscle's volume as in claim 15, wherein the skeletal muscle is a tricep.
